(19) United States
(12) Patent Application Publication
(10) Pub. No.: US 2015/0246009 A1
(43) Pub. Date: Sep. 3, 2015

(54) Stable injectable pharmaceutical composition of epinephrine or salts thereof

(71) Applicant: WOCKHARDT LIMITED, Aurangabad (IN)

(72) Inventors: Amit Gupta, Faridabad 2, (IN); Rajendra Nandlal Nagori, Aurangabad 0, (IN); Arvind Yekanathsa Merwade, Belgaum 582102, (IN); Keshav Deo, Vadodara 1, (IN); Girish Kumar Jain, Delhi 4, (IN)

(73) Assignee: WOCKHARDT LIMITED, Aurangabad 0, (IN)

(21) Appl. No.: 14/430,423

(22) PCT Filed: May 3, 2013
(86) PCT No.: PCT/IB2013/053520

§ 371 Date: Mar. 23, 2015

(30) Foreign Application Priority Data
Oct. 8, 2012 (IN) 2934/MUM/2012

(51) Int. Cl. A61K 31/137; A61K 9/00; A61K 47/02

(52) U.S. Cl. CPC A61K 31/137; A61K 47/02; A61K 9/0019

(57) ABSTRACT

The present invention refers to a stabilized injectable pharmaceutical composition of epinephrine or salts thereof comprising sodium metabisulfite, wherein the ratio of the amount of epinephrine or salt thereof to sodium metabisulfite in the composition ranges from about 1:0.005 to about 1:1.5 by weight. It has been observed that stable injectable epinephrine compositions with excellent storage stability and substantially free of overages can be prepared using sodium metabisulfite in said ratios. Particularly, it was found that using sodium metabisulfite in said ratios controls levels of adrenaline sulfonate impurity in said pharmaceutical compositions.

### FIELD OF THE INVENTION

The present invention relates to a stabilized injectable pharmaceutical composition of epinephrine or salts thereof comprising sodium metabisulfite, wherein the ratio of the amount of epinephrine or salt thereof to sodium metabisulfite in the composition ranges from about 1:0.005 to about 1:1.5 by weight. It has been observed that stable injectable epinephrine compositions with excellent storage stability and substantially free of overages can be prepared using sodium metabisulfite in said ratios. Particularly, it was found that using sodium metabisulfite in said ratios controls levels of adrenaline sulfonate impurity in said pharmaceutical compositions.

### BACKGROUND OF THE INVENTION

Epinephrine, also known as adrenaline, is a sympathomimetic catecholamine. Chemically, epinephrine is B-(3,4dihydroxyphenyl)-a-methyl-amino ethanol.

Epinephrine is the drug of choice for the initial treatment of anaphylaxis. Many epinephrine products are commerically available currently. For instance, epinephrine is marketed in the United States in the form of intramuscular and subcutaneous injection under trade name Twinject®, Auvi-Q®, EpiPen® Auto-Injector, which contains 0.3 mg epinephrine, and EpiPen® Jr Auto-Injector, which contains 0.15 mg epinephrine.

It is well known in the art that there is an issue with potency of epinephrine (both in free base form and ionic form) when used in presence of oxygen and free radicals i.e degradation of epinephrine is accelerated in the presence of oxygen and free radicals.

Numerous studies have been conducted to address the effect of formulation variables on the epinephrine degradation kinetics, and attempts have been made to improve the formulation stability.

U.S. Pat. No. 3,149,035 discloses use of bisulphite and boric acid to enhance stability of the catechol amines.

U.S. Pat. No. 3,966,905 discloses catecholamine solutions at mild pH are suitable for physiological use.

Several literatures suggests that there is an increase in stability of epinephrine when stored in gas-tight containers with an inert gas (e.g. nitrogen) purging, and/or limiting or protecting the epinephrine formulation from direct light exposure or storing in a secondary opaque package. Inspite of using sodium metabisulfite controlling the degradation of epinephrine however continues to be an issue. In addition, interaction of sodium metabisulfite with epinephrine further leads to complications.

Currently marketed products of epinephrine as discussed above includes sodium metabisulfite as an antioxidant as it prevents degradation of the product due to oxidation that may take place during manufacturing, filling, storage, and environmental influence on the formulation.

Currently marketed products of epinephrine i.e EpiPen® Auto-Injector containing 0.3 mg epinephrine and EpiPen® Jr Auto-Injector containing 0.15 mg of epinephrine comprises of same amount of sodium metabisulfite i.e. 0.5 mg. It is also observed that EpiPen® Jr Auto-Injector product generally degrades relatively faster than that in EpiPen® Auto-Injector, presumably due to exposure of the product to substantial vacant space left in the cartridge.

Moreover, Currently marketed products of epinephrine i.e the EpiPen® Auto-Injector products contains more than about 20% of the epinephrine overages. This is in order to compensate the amount of epinephrine degraded during manufacture or over storage. Such overages however, may either lead to undesirable side effects due to dose inaccuracy or generate more degradation products in the product.

Further, currently marketed products contain 0.3 mg or 0.15 mg in 2.0 ml solution, of which only 0.3 ml is injected and rest 1.7 ml is discarded, which leads to lots of wastage.

Since said product is used in severe anaphylactic reactions, so controlling impurities and improvement of stability is critical. Hence, there exists an enduring need for improved and stable pharmaceutical composition of epinephrine, which exhibits excellent storage stability and does not require addition of epinephrine overages.

### SUMMARY OF THE INVENTION

In one aspect, the present invention provides a stabilized injectable pharmaceutical composition comprising epinephrine or salt thereof and sodium metabisulfite, wherein the ratio of the amount of epinephrine or salt thereof to sodium metabisulfite in the composition ranges from about 1:0.005 to about 1:1.5 by weight.

In another aspect, the present invention provides a stabilized injectable pharmaceutical composition comprising epinephrine or salt thereof and sodium metabisulfite, wherein the ratio of the amount of epinephrine or salt thereof to sodium metabisulfite in the composition ranges from about 1:0.005 to about 1:1.5 by weight characterized in that said composition contains epinephrine having purity equal to or greater than 98%.

In another aspect, the present invention provides a stabilized injectable pharmaceutical composition comprising epinephrine or salt thereof and sodium metabisulfite, wherein the ratio of the amount of epinephrine or salt thereof to sodium metabisulfite in the composition ranges from about 1:0.005 to about 1:1.5 by weight characterized in that said composition contains total impurity of 4% or less.

In another aspect, the present invention provides a stabilized injectable pharmaceutical composition comprising epinephrine or salt thereof and sodium metabisulfite, wherein the ratio of the amount of epinephrine or salt thereof to sodium metabisulfite in the composition ranges from about 1:0.005 to about 1:1.5 by weight characterized in that said composition contains no single impurity of greater than 3%.

In another aspect, the present invention provides a stabilized injectable pharmaceutical composition comprising epinephrine or salt thereof and sodium metabisulfite, wherein the ratio of the amount of epinephrine or salt thereof to sodium metabisulfite in the composition ranges from about 1:0.005 to about 1:1.5 by weight characterized in that said composition contains adrenaline sulfonate impurity of about 3.0% or less at RRT 0.15.

In another aspect, the present invention provides a stabilized injectable pharmaceutical composition comprising epinephrine or salt thereof and sodium metabisulfite, wherein the ratio of the amount of epinephrine or salt thereof to sodium metabisulfite in the composition ranges from about 1:0.005 to about 1:1.5 by weight characterized in that said composition contains noradrenaline impurity of about 0.1% or less.

In another aspect, the present invention provides a stabilized injectable pharmaceutical composition comprising epinephrine or salt thereof and sodium metabisulfite, wherein the ratio of the amount of epinephrine or salt thereof to sodium metabisulfite in the composition ranges from about 1:0.005 to about 1:1.5 by weight characterized in that said composition contains adrenalone impurity of about 0.5% or less.

In another aspect, the present invention provides a stabilized injectable pharmaceutical composition comprising epinephrine or salt thereof and sodium metabisulfite, wherein the ratio of the amount of epinephrine or salt thereof to sodium metabisulfite in the composition ranges from about 1:0.005 to about 1:1.5 by weight characterized in that said composition contains N-benzyl adrenalone impurity of about 0.1% or less.

In another aspect, the present invention provides a stabilized injectable pharmaceutical composition comprising epinephrine or salt thereof and sodium metabisulfite, wherein the ratio of the amount of epinephrine or salt thereof to sodium metabisulfite in the composition ranges from about 1:0.005 to about 1:1.5 by weight characterized in that said composition contains impurity observed at RRT 0.17, RRT 0.2, or RRT 0.73 of about 0.5% or less.

In another aspect, the present invention provides a stabilized injectable pharmaceutical composition comprising epinephrine or salt thereof and sodium metabisulfite, wherein the ratio of the amount of epinephrine or salt thereof to sodium metabisulfite in the composition ranges from about 1:0.005 to about 1:1.5 by weight characterized in that said composition contains retains at least 90% w/w of total potency of epinephrine after storage at 25° C. and 60% relative humidity for at least 3 months.

In another aspect, the present invention provides a stabilized injectable pharmaceutical composition comprising epinephrine or salt thereof, sodium metabisulfite, one or more tonicity-adjusting agents, one or more pH adjusting agents, aqueous vehicle, and optionally one or more other pharmaceutically acceptable excipients, wherein the ratio of the amount of epinephrine or salt thereof to sodium metabisulfite in the composition ranges from about 1:0.005 to about 1:1.5 by weight.

In another aspect, the present invention provides a process for preparation of stable injectable pharmaceutical composition comprising epinephrine or salt thereof, which process comprises of mixing epinephrine or salt thereof, sodium metabisulfite, aqueous vehicle, and optionally one or more other pharmaceutically acceptable excipients, wherein the ratio of the amount of epinephrine or salt thereof to sodium metabisulfite in the composition ranges from about 1:0.005 to about 1:1.5 by weight.

In another aspect, the present invention provides use of a stable injectable pharmaceutical composition comprising epinephrine or salt thereof and sodium metabisulfite, wherein the ratio of the amount of epinephrine or salt thereof to sodium metabisulfite in the composition ranges from about 1:0.005 to about 1:1.5 by weight for the preparation of medicaments useful for treating allergic reactions (Type I) including anaphylaxis to stinging insects and biting insects, allergen immunotherapy, foods, drugs, diagnostic testing substances and other allergens, as well as idiopathic anaphylaxis or exercise-induced anaphylaxis, comprising administering to human patient in need thereof.

In another aspect, the present invention provides a stabilized injectable pharmaceutical composition comprising 0.3 mg of epinephrine or salt thereof and 0.3 mg of sodium metabisulfite in 0.3 ml of solution, wherein the said 0.3 ml of solution is delivered completely in single injection.

In another aspect, the present invention provides a stabilized injectable pharmaceutical composition comprising 0.15 mg of epinephrine or salt thereof and 0.3 mg of sodium metabisulfite in 0.3 ml of solution, wherein the said 0.3 ml of solution is delivered completely in single injection.

Embodiments of the pharmaceutical composition may include one or more of the following features. For example, the pharmaceutically acceptable excipients may include solubilizers, anti-oxidants, buffering agents, pH adjusting agents, co-solvents, chelating agents, stabilizers, preservatives, lubricants, tonicity adjusting agents, cryoprotectants and the like known to the art used either alone or in combination thereof.

### DETAILED DESCRIPTION OF THE INVENTION

The inventors of the present invention have surprisingly found that while making an injectable composition of epinephrine, amount of sodium metabisulfite plays a critical role in order to control degradation due to oxidation as well as in controlling impurities. It was surprisingly found that epinephrine reacts with sodium metabisulfite, thereby leading to formation of adrenaline sulfonate impurity. In particular, the inventors have found that judicial amount of sodium metabisulfite can effectively curb the oxidation of epinephrine and eventually control generation of sulfonate impurity along with a wide range of several other epinephrine impurities. As a result, inventors of the present invention have found a novel way of preparing the injectable pharmaceutical composition of epinephrine which can exhibit excellent storage stability.

The inventors of the present invention further surprisingly found that judicial amount of sodium metabisulfite can retain epinephrine potency in the composition during the manufacture as well as over the storage period, thus may eliminate the need of adding epinephrine overages.

The present invention relates to novel and stabilized injectable pharmaceutical compositions of epinephrine and process of preparing such compositions.

The stabilized injectable pharmaceutical composition of the present invention comprises epinephrine or salt thereof and sodium metabisulfite, characterized in that the ratio of the amount of epinephrine or salt thereof to sodium metabisulfite in the composition ranges from about 1:0.005 to about 1:1.5 by weight.

The stabilized injectable pharmaceutical composition of the present invention comprises epinephrine or salt thereof and sodium metabisulfite, characterized in that the composition is substantially free of epinephrine overages.

The term “epinephrine” used throughout the specification refers to not only epinephrine per se, but also its pharmaceutically acceptable salts, pharmaceutically acceptable solvates, pharmaceutically acceptable hydrates, pharmaceutically acceptable enantiomers, pharmaceutically acceptable derivatives, pharmaceutically acceptable polymorphs and pharmaceutically acceptable prodrugs thereof.

The term “substantially free” used throughout the specification refers to pharmaceutical compositions of epinephrine comprising less than about 10% by weight of epinephrine overages.

The “stabilized injectable pharmaceutical composition” of the present invention refers to injectable compositions characterized by epinephrine having purity equal to or greater than 98% by weight or total impurity of 4% or less or no single impurity of greater than 3% or adrenaline sulfonate impurity of about 3.0% or less at RRT 0.15 or noradrenaline impurity of about 0.1% or less or adrenalone impurity of about 0.5% or less or N-benzyl adrenalone impurity of about 0.1% or less.

In an embodiment, the stabilized injectable pharmaceutical composition of the present invention retains at least 90% w/w of total potency of epinephrine after storage at 25° C. and 60% relative humidity for at least 3 months.

Extent of the stability have been observed to be more pronounced on four specific impurities (RRT 0.15, RRT 0.17, RRT 0.20, and RRT 0.73) out of a large number of impurities that affect the stability not only in the presence or absence of sodium metabisulfite but also under normal atmospheric condition, or inert atmosphere, or the exposure to oxygen. Some of the specific impurities formed which had a significant influence on the stability are as given below.

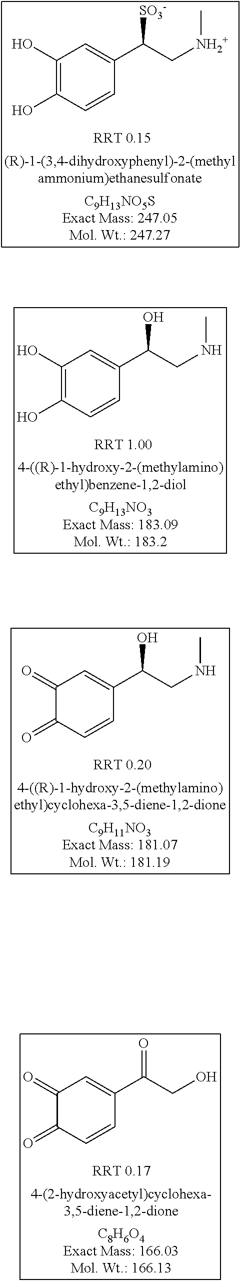

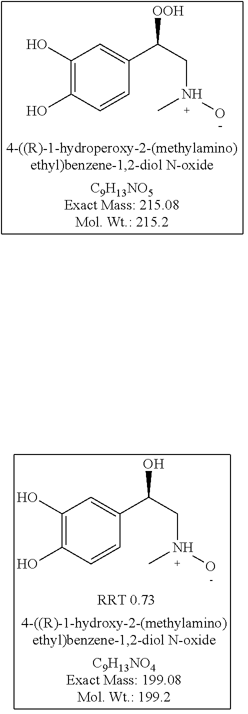

In an embodiment, the stabilized injectable pharmaceutical composition comprises total impurity of about 4% or less when stored at 25° C. and 60% relative humidity for at least 3 months.

In another embodiment, the stabilized injectable pharmaceutical composition comprises adrenaline sulfonate impurity of about 3% or less when stored at 25° C. and 60% relative humidity for at least 3 months.

In a further embodiment, the stabilized injectable pharmaceutical composition comprises noradrenaline impurity of about 0.05% or less when stored at 25° C. and 60% relative humidity for at least 3 months.

In a further embodiment, the stabilized injectable pharmaceutical composition comprises adrenalone impurity of about 0.3% or less when stored at 25° C. and 60% relative humidity for at least 3 months.

In a further embodiment, the stabilized injectable pharmaceutical composition comprises N-benzyl adrenalone impurity of about 0.05% or less when stored at 25° C. and 60% relative humidity for at least 3 months.

In a further embodiment, the stabilized injectable pharmaceutical composition comprises impurity observed at RRT 0.17, RRT 0.20, or RRT 0.73 of about 0.5% or less when stored at 25° C. and 60% relative humidity for at least 3 months.

Various methods of analyzing (characterization and quantification) the impurities are well established in the art. Various spectoroscopic techniques, such as NMR, MS, IR etc. and chromatographic techniques, such as HPLC, HPLC-TLC, HPLC-CE and hyphenated methods, such as LC-MS-MS, HPLC-DAD-MS, HPLC-NMR, GC-MS & LC-MS can be used for analyzing impurities.

Related substances of Epinephrine were performed by reverse phase chromatography using Cosmosil AR-II, C-18, (250×4.6) mm, 5 μm columns. All impurities were separated in gradient mode with resolution more than 3.0. The detection was carried out at optimum wavelength 210 nm

The pharmaceutical composition of the present invention may be developed in the form of a dosage form suitable of parenteral administration. The parenteral route of administration of the compositions comprises subcutaneous, intramuscular, intravenous, transdermal, intradermal, intranasal, intraarterial and intraperitoneal injection or infusion. In an embodiment the injection includes aqueous vehicle based injection and oil based injection (e.g. depot injection).

The pharmaceutical composition of the present invention further comprises various pharmaceutically acceptable excipients suitable for parenteral administration. Such excipient includes, but not limited to pH adjusting agents or buffers, co-solvents, chelating agents, isotonicity adjusting agents, preservatives, and aqueous vehicle.

Examples of suitable pH adjusting agents includes, but not limited to hydrochloric acid, citric acid, ascorbic acid, acetic acid, tartaric acid, phosphoric acid, metaphosphoric acid, polymetaphosphoric acid, carbonic acid, sodium hydroxide, potassium hydroxide, sodium citrate, potassium citrate, sodium bicarbonate, potassium bicarbonate, ammonium carbonate, sodium hydrogen phosphate, potassium hydrogen phosphate, ethanolamine, diethanolamine, triethanolamine, hexane-1,2-diamine, sodium carbonate, sodium potassium tartrate, potassium metaphosphate, potassium polymetaphosphate, and sodium metaphosphate. The pH of the pharmaceutical composition preferably ranges from 2.2 to 5.0.

Examples of suitable buffers includes, but not limited to pharmaceutically acceptable salts and acids of acetate, glutamate, citrate, tartrate, benzoate, lactate, histidine or other amino acids, gluconate, phosphate, malate, succinate, formate, propionate, and carbonate.

Examples of suitable co-solvents includes, but not limited to ethanol, glycerol, propylene glycol, polyethylene glycol, and different oils.

Examples of suitable chelating agents includes, but not limited to calcium ethylenediaminetetraacetic acid (EDTA), calcium diethylenetriaminepentaacetic acid (DTPA), calcium hydroxyethylenediaminetriacetic acid (HEDTA), calcium ethylene glycol-bis-(2-aminoethyl)-N,N,N′,N′-tetraacetic acid (EGTA), calcium nitrilotriacetic acid (NTA), calcium citrate, and calcium salt derivatives thereof.

Examples of suitable isotonicity adjusting agents includes, but not limited to anhydrous or hydrous forms of sodium chloride, dextrose, sucrose, xylitol, fructose, glycerol, sorbitol, mannitol, potassium chloride, mannose, calcium chloride, magnesium chloride and other inorganic salts.

The pharmaceutical composition of the present invention may be hypotonic, isotonic or hypertonic. In an embodiment, the pharmaceutical composition have a tonicity from about 250 to about 350 mOsm/kg.

Examples of suitable preservative include, but not limited to benzyl alcohol, propyl and methyl paraben.

The present invention also provides for a process of manufacturing aqueous epinephrine compositions. The process involves mixing epinephrine, sodium metabisulfite, water, and optionally other pharmaceutically acceptable excipients together.

The composition may be rendered non-pyrogenic, if required, by passing through Tangential Flow Filtration System (TFF) before sterilization. The composition may be sterilized by membrane filter of 0.22 μm pore size.

The process of manufacturing the aqueous epinephrine compositions of the present invention further may comprises sterilization of the composition. The compositions may be sterilized by known and acceptable methods. In an embodiment, the composition is sterilized by filtering through a sterilizing grade filter. Preferably, the solution is filtered through 0.2 μm sterilizing grade filters.

After sterilization, it may be desirable to aseptically place the filtered solutions into sterile containers such as vials, ampoules, or cartridges of the pre-filled syringes. In an embodiment, after aseptically placing the filtered solution into the cartridge of prefilled syringes, the air in the cartridge is purged with an inert gas, such as nitrogen, and then the filled cartridge is sealed in the pre-filled syringe.

The present invention further refers to the use of the above defined composition for the preparation of medicaments useful for treating allergic reactions (Type I) including anaphylaxis to stinging insects (e.g., order Hymenoptera, which include bees, wasps, hornets, yellow jackets and fire ants) and biting insects (e.g., triatoma, mosquitos), allergen immunotherapy, foods, drugs, diagnostic testing substances (e.g., radiocontrast media) and other allergens, as well as idiopathic anaphylaxis or exercise-induced anaphylaxis.

The present invention is further illustrated by the following examples which are provided merely to be exemplary of the invention and do not limit the scope of the invention. Certain modifications and equivalents will be apparent to those skilled in the art and are intended to be included within the scope of the present invention.

### Example 1

### Epinephrine Injection 0.3 mg/0.3 mL

TABLE 1

| Sr. | | Composition A | Composition B |
|---|---|---|---|
| No. | Ingredient | Qty. (% w/v) | Qty. (% w/v) |
| 1. | Epinephrine | 0.3 | 0.3 |
| 2. | Sodium chloride | 1.8 | 1.8 |
| 3. | Sodium | 0.3 | 0.2 |
| | metabisulfite | | |
| 4. | 1N HCl Solution | q.s. to pH 2.2-5.0 | q.s. to pH 2.2-5.0 |
| 5. | Water for Injection | q.s. to 0.3 mL | q.s. to 0.3 mL |

### Example 2

### Epinephrine Injection 0.15 mg/0.3 mL

TABLE 2

| Sr. | | Composition C | Composition D |
|---|---|---|---|
| No. | Ingredient | Qty. (% w/v) | Qty. (% w/v) |
| 1. | Epinephrine | 0.15 | 0.15 |
| 2. | Sodium chloride | 1.8 | 1.8 |
| 3. | Sodium | 0.15 | 0.10 |
| | metabisulfite | | |
| 4. | 1N HCl Solution | q.s. to pH 2.2-5.0 | q.s. to pH 2.2-5.0 |
| 5. | Water for Injection | q.s. to 0.3 mL | q.s. to 0.3 mL |

Process:

Sodium chloride was dissolved in water for injection under continuous nitrogen sparging. 1N HCl solution was added to adjust the pH. Epinephrine and sodium metabisulfite were sequentially added to the solution under stirring to get clear solution. Final volume of the solution was made with water for injection. pH of the final solution can be adjusted using HCl solution if required. The solution was then subjected to filtration through 0.22μ membrane filter. The solution was then filled in sterile 1 mL pre-filled syringes.

### Example 3

### Comparative Stability Study of Effect of Sodium Metabisulfite Concentration on Stability of Epipen/Epipen Jr. v Epinephrine Composition of the Invention

TABLE 3

| | Composition 1 | Composition 2 | Composition 3 | Composition 4 |
|---|---|---|---|---|
| | (Epipen Jr) | (Invention) | (Epipen) | (Invention) |
| | 0.15 mg | 0.15 mg | 0.3 mg | 0.3 mg |
| | Epinephrine, | Epinephrine, | Epinephrine, | Epinephrine, |
| | 0.5 mg | 0.3 mg | 0.5 mg | 0.3 mg |
| | Sodium | Sodium | Sodium | Sodium |
| | metabisulfite | metabisulfite | metabisulfite | metabisulfite |
| Description | Clear colorless | Clear colorless | Clear colorless | Clear colorless |
| | Solution | Solution | Solution | Solution |
| Storage | 25° C., 60% | 25° C., 60% | 25° C., 60% | 25° C., 60% |
| Conditions | RH for 3 months | RH for 3 months | RH for 3 months | RH for 3 months |
| Assay | 107.8 | 112 | 108.8 | 115.5 |
| Adrenaline | 4.983 | 0.415 | 3.893 | 2.721 |
| sulfonate | | | | |
| impurity (%) | | | | |
| Noradrenaline | 0.0 | 0.0 | 0.0 | 0.0 |
| impurity (%) | | | | |
| Adrenalone | 0.128 | 0.023 | 0.071 | 0.083 |
| impurity (%) | | | | |
| N-Benzyl | 0.0 | 0.0 | 0.0 | 0.0 |
| adrenalone | | | | |
| impurity (%) | | | | |
| Total impurity | 6.288 | 0.935 | 4.65 | 3.633 |
| (%) | | | | |

Result of the stability study conducted on the composition of the present invention (Composition 2 & 4) indicates that epinephrine composition containing 0.3 mg sodium metabolite exhibits excellent storage stability relative to epinephrine composition containing 0.5 mg sodium metabolite over the storage period.

1. A stabilized injectable pharmaceutical composition of epinephrine or salt thereof comprising sodium metabisulfite, wherein the ratio of the amount of epinephrine or salt thereof to sodium metabisulfite in the composition ranges from about 1:0.005 to about 1:1.5 by weight, characterized in that said composition contains epinephrine having purity equal to or greater than 98% by weight.

2. A stabilized injectable pharmaceutical composition of epinephrine comprising sodium metabisulfite, wherein the ratio of the amount of epinephrine or salt thereof to sodium metabisulfite in the composition ranges from about 1:0.005 to about 1:1.5 by weight, characterized in that said composition contains total impurity of less than 4%.

3. A stabilized injectable pharmaceutical composition of epinephrine comprising sodium metabisulfite, wherein the ratio of the amount of epinephrine or salt thereof to sodium metabisulfite in the composition ranges from about 1:0.005 to about 1:1.5 by weight, characterized in that said composition contains no single impurity of greater than 3%.

4. A stabilized injectable pharmaceutical composition of epinephrine or salt thereof comprising sodium metabisulfite, wherein the ratio of the amount of epinephrine or salt thereof to sodium metabisulfite in the composition ranges from about 1:0.005 to about 1:1.5 by weight, characterized in that said composition is substantially free of epinephrine overages.

5. The stabilized injectable pharmaceutical composition of claim 1, wherein the composition comprises adrenaline sulfonate less than about 3.0% or less at RRT 0.15.

6. The stabilized injectable pharmaceutical composition of claim 1, wherein the composition comprises less than about 0.5% impurity observed at RRT 0.17, RRT 0.2, or RRT 0.73.

7. A stabilized injectable pharmaceutical composition comprising epinephrine, sodium metabisulfite, wherein the ratio of the amount of epinephrine or salt thereof to sodium metabisulfite in the composition ranges from about 1:0.005 to about 1:1.5 by weight, characterized in that said composition retains at least 90% w/w of total potency of epinephrine after storage at 25° C. and 60% relative humidity for at least 3 months.

8. A stabilized injectable pharmaceutical composition comprising 0.3 mg of epinephrine or salt thereof and 0.3 mg of sodium metabisulfite in 0.3 ml of solution, wherein the said 0.3 ml of solution is delivered completely in single injection.

9. A stabilized injectable pharmaceutical composition comprising 0.15 mg of epinephrine or salt thereof and 0.3 mg of sodium metabisulfite in 0.3 ml of solution, wherein the said 0.3 ml of solution is delivered completely in single injection.
